(19) United States
(12) Patent Application Publication
(10) Pub. No.: US 2009/0275829 A1
(43) Pub. Date: Nov. 5, 2009

(54) Contrast media injector kit

(76) Inventors: Mahesh Agarwal, Markham (CA); Stephen Romaniuk, Mississauga (CA)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 12/383,262

(22) Filed: Mar. 20, 2009

(30) Foreign Application Priority Data
Apr. 30, 2008 (CA) 2,630,561

(51) Int. Cl. A61M 5/00; A61B 6/00

(52) U.S. Cl. 600/433

(57) ABSTRACT

There is provided an integrated injector subassembly or kit. The integrated injector kit comprises: Y-tubing comprising an output arm that is for releasable connection with a patient line and first and second input arms that each independently and integrally connect with an output port of first and second fluid control valves; each fluid control valve comprising: (a) the output port that is integrally connected to one of the first and second input arms of the Y-tubing; (b) an inlet port for connection with a container of a contrast agent or saline and for receiving the contrast agent or saline; and (c) a syringe port for connection with a syringe; and each pairing of fluid control valve and input arm of the Y-tubing defining a fluid path comprising at least one back check valve.

### RELATED APPLICATIONS

This application claims the benefit of Canadian Patent Application No. 2,630,561 filed Apr. 30, 2008, the disclosure of which is incorporated herein by reference in its entirety.

### FIELD OF THE INVENTION

The present invention relates to a injector for medical imaging techniques.

More particularly, the present invention relates to a multi-dosing contrast media injector subassembly or kit for medical imaging techniques.

### BACKGROUND OF THE INVENTION

Structural imaging, for example in the areas of computerized tomography (CT), magnetic resonance imaging (MRI), and ultrasound imaging, is an evolving field, with the amount of time required to obtain an image, the image quality, and patient comfort acting as the driving forces behind the technological advancements.

CT is a specialized x-ray procedure. The procedure can be used to examine almost any part of the body. CT involves using an x-ray tube that rotates around the patient's body and special detectors that collect the x-ray information that is produced. A computer converts the information gathered into cross-sectional pictures or “slices” of the body that are then viewed on a television monitor. The procedure is often referred to as CT “scan”. The pictures are photographed onto a film. CT usually provides more information than regular x-rays. Since the images are created by a computer, they can be manipulated or changed to show specific areas of interest more clearly. These images are used to help diagnose conditions that may not show up on conventional x-rays.

MRI is primarily used in medical imaging to visualise the structure and function of the body. It provides detailed images of the body in any plane. MR has much greater soft tissue contrast than CT making it especially useful in neurological, musculoskeletal, cardiovascular and oncolological diseases. Unlike conventional x-ray examinations and CT scans, MRI does not depend on ionizing radiation. Instead, radio waves are directed at protons, the nuclei of hydrogen atoms, in a strong magnetic field.

Ultrasound imaging (also known as medical sonography or ultrasonography) is widely used to perform diagnosis or therapeutic procedures, for example biopsies or drainage of fluid collections. Sonographers are medical professionals who perform scans for diagnostic purposes. Sonographers typically use a hand-held probe (i.e. a transducer) that is placed directly on and moved over the patient. A water-based gel is used to couple the ultrasound between the transducer and patient. Sonography is effective for imaging soft tissues of the body. Superficial structures such as muscles, tendons, testes, breast and the neonatal brain are imaged at a higher frequency (7-18 MHz), which provides better axial and lateral resolution. Deeper structures such as liver and kidney are imaged at a lower frequency 1-6 MHz with lower axial and lateral resolution but greater penetration.

Contrast media may be used in many medical diagnostic and therapeutic imaging procedures. Diagnostically these include, for example, X-ray procedures, CT scanning, MRI, and ultrasound imaging. Contrast media may also be used during therapeutic procedures such as angioplastic and other interventional radiologic procedures.

Contrast is a key factor in perceiving a difference in the density between areas of a radiographic image. Therefore, radiographic contrast media are instrumental in enhancing the contrast among various body tissues thereby improving evaluation of the pathologies in the body. The general strategy in designing any contrast media relies upon exploiting the differences in contrast media distribution so as to achieve opacification of specific tissues of interest in relation to the background tissue. An ideal contrast medium has innumerable criteria to meet before it is suitable for administration in the body. Since contrast media use is integral to all imaging methods, its development has paralleled advances in state-of-the-art imaging technology.

The use of contrast agents and injectors has played a major role in the progression of quality, timing, and care. Contrast injector manufacturers have concentrated on developing injectors that deliver contrast at sufficient rates while allowing for additional timing and safety capabilities, such as saline flush, saline test inject, and extravasation detection. A variety of companies manufacture a range of contrast injectors suited for specific CT, MRI, or ultrasound techniques, with varying capabilities and features. Contrast agent manufacturers also have focused on developing products that are matched to specific techniques and target areas, while reducing patients' potential pain and burning sensations.

There are two types of contrast injectors available in the market. A single syringe system commonly called the single-head injector; and a dual-syringe system called the dual-head injector. Dual-head injectors are currently being used for more advanced imaging applications requiring the use of a saline chase as the added saline injector provides a tighter bolus and reduced contrast volume. The dual-head injector system pushes a column of saline after the contrast to keep the contrast bolus tight and flowing at the right speed, thus creating an even distribution of contrast while reducing the amount of contrast load to the patient and the reduction of cost. Also, the dual-head injector allows the ability to test the injection site with saline prior to the injection of contrast to ensure the patency of the vein.

A typical set-up of a dual-head injector involves two syringes, one for contrast and one for saline. The syringes can come preloaded with media, contrast or saline, or connections between a syringe and a media bottle or bag may be established. The two syringes are connected to a patient manifold that provides an outlet for the contrast or saline to be delivered to the patient through a patient line. Fluid control valves, such as manual stopcock valves, back check valves, dual-check valves and the like may be placed up or downstream of the patient manifold.

For fear of cross contamination between patients, a prevalent approach in imaging techniques is to connect and create a new injector setup for each new patient. This is an expensive and time consuming setup.

Furthermore, the amount of contrast given to each patient is a fraction of the total volume of contrast that may be contained in the syringe, the bottle or the bag. With a new setup for each patient the unused contrast is discarded. This proves to be very costly as the contrast media is very expensive.

To contain the healthcare costs, Health Canada conducted a study in 1996 (Infection Control—Preliminary Report: Biosafety Analysis of One Way Backflow Valves for Multiple Patient Use of Low Osmolar Intravenous Contrast Solution; Volume 22-04, 15 Feb. 1996, Public Health Agency of Canada) to determine if multiple patient dosing of expensive contrast media for achieving cost reductions is a safe and viable option. They concluded that transfer devices with appropriately designed and tested check valves can prevent the backflow of potentially contaminated fluids in a multi-dosing setting of contrast media, and therefore, multi-dosing is indeed a safe option.

With multi-dosing the dual-head injector set-up is used typically for up to four hours, and only the patient line is changed for each new patient. At the end of four hours the entire set-up is changed.

Multi-dosing has been in use in Canada for the last number of years, and more hospitals are adopting multi-dosing in order to save costs in material and labour.

A common problem with current techniques of both single dosing and multi-dosing is the number of connections, the complexity of set-up, the mismatch amongst the components supplied by different vendors and the associated costs involved.

### SUMMARY OF THE INVENTION

In an aspect, there is provided an integrated contrast injector kit comprising:

Y-tubing comprising an output arm that is for releasable connection with a patient line and first and second input arms that each independently and integrally connect with an output port of first and second fluid control valves;

each fluid control valve comprising: (a) the output port that is integrally connected to one of the first and second input arms of the Y-tubing; (b) an inlet port for connection with a container of a contrast agent or saline and for receiving the contrast agent or saline; and (c) a syringe port for connection with a syringe; and

each pairing of fluid control valve and input arm of the Y-tubing defining a fluid path comprising at least one back check valve.

### BRIEF DESCRIPTION OF THE DRAWINGS

Embodiments will now be described, by way of example only, with reference to the attached Figures, wherein:

FIG. 1a is a perspective view of an unassembled CT contrast injector set-up using a stopcock as a fluid control valve;

FIG. 1b is a perspective view of an unassembled CT contrast injector set-up using a dual check valve as a fluid control valve;

FIG. 2a is a perspective view of an assembled CT contrast injector set-up using a dual check valve as a fluid control valve showing, in between the solid white lines, a subassembly of components that are integrally connected and may be used for multi-dosing with additional components that are meant for releasable connection with the subassembly being located outside of the solid white lines;

FIG. 2b is a perspective view of an assembled CT contrast injector set-up subassembly using a stopcock as a fluid control valve showing components that are integrally connected and may be used for multi-dosing;

FIG. 3 is a perspective view of an assembled MRI contrast injector set-up subassembly using a dual check valve as a fluid control valve showing components that are integrally connected and may be used for multi-dosing.

### DETAILED DESCRIPTION OF THE EMBODIMENTS

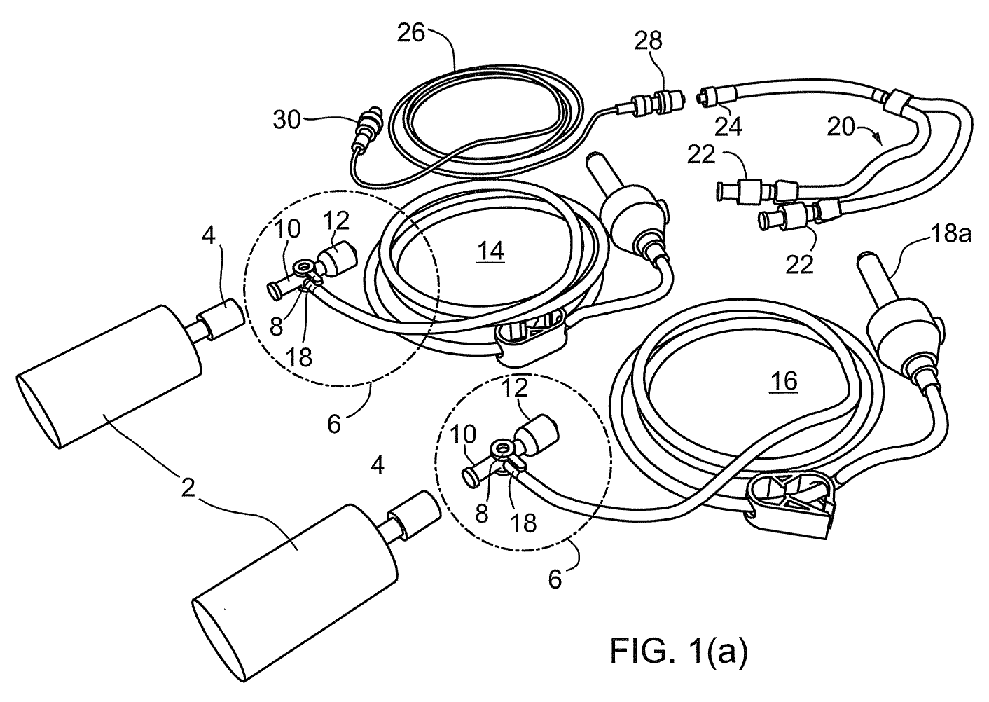

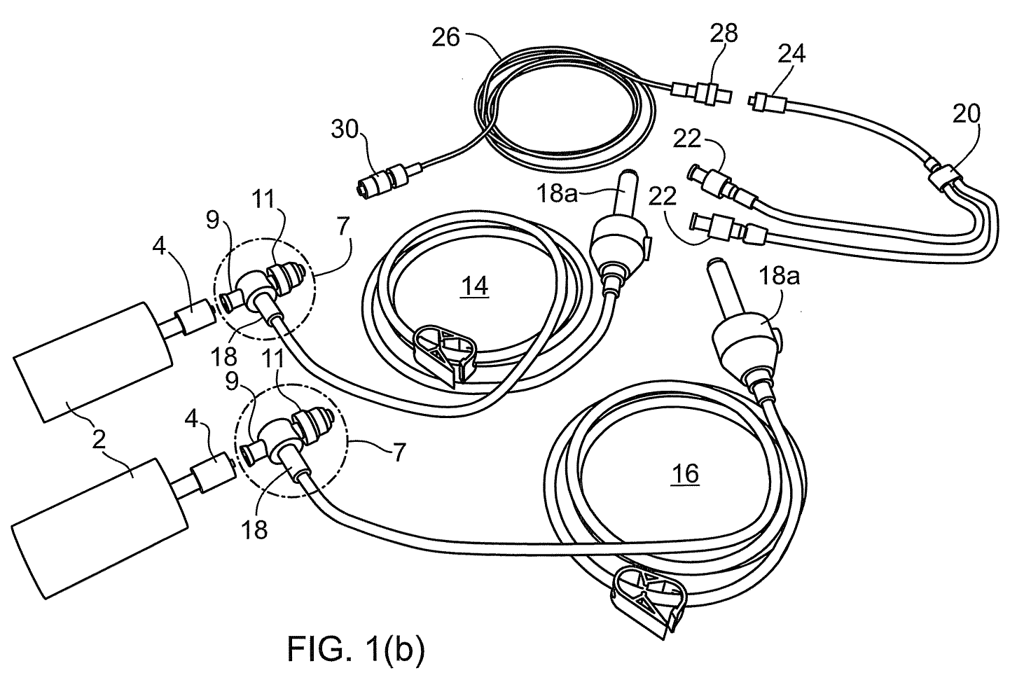

FIGS. 1a and 1b show components of a dual-head injector tubing set-up that can be used for CT injections. The dual-head injector has two syringes (2), one for contrast and one for saline. With respect to each of FIGS. 1a or 1b the two syringes are each connected to a 3-port tubing set (i.e., saline 3-port tubing set 14, and contrast 3-port tubing set 16) each comprising a fluid control valve. Flow of fluid through the three ports of the 3-port tubing set is controlled by the fluid control valve, for example a manually operated 3-way stopcock valve (6 in FIG. 1a) or an automatic dual check valve (7 in FIG. 1b). The syringe port of the tubing set (10 in FIG. 1a and 9 in FIG. 1b) is a female luer port that connects to the male luer port (4) of the syringe in the injector system; the inlet port of the tubing set (18) connects to a bag or a bottle (not shown) of either contrast or saline via a vented spike (18a), and the output port of the 3-port tubing set (12 in FIGS. 1a and 11 in FIG. 1b) is a male luer port that provides an outlet for the contrast or saline to be delivered through Y-connector (20) and patient line (26) to the patient.

The terms Y-connector and Y-tubing are used interchangeably and are meant to indicate two input arms that converge into an output arm. The input arms receive fluid at an input port and the fluid flows through the output arm and exits from an output port.

In the stopcock set-up shown in FIG. 1a the operator manually turns the stopcock handle (8) to select the mode of operation and thereby control flow of fluid. In one position the syringe fills from the contrast or saline, while in another position the syringe pushes the contrast or saline to the patient.

In the dual check valve set-up shown in FIG. 1b the mode of operation to control fluid flow is selected automatically. A check valve is a mechanical device, a valve, that normally allows fluid to flow through it in only one direction. There is no reverse flow. In check valves the cracking pressure is the minimum upstream pressure at which the valve will operate. The dual check valve (7) comprises a first check valve positioned at inlet port (18) that allows contrast or saline to flow into the syringe (2), and a second check valve positioned at output port (11) that allows fluid to flow from the syringe (2) to the patient line (26). The dual check valve arrangement (7) allows a user to fill the syringe (2) from the bag or bottle of contrast or saline, and then use the syringe to push fluid to the patient line (26) without having to manually control fluid flow as in the stopcock arrangement. Thus, the use of the dual check valve allows for automation or computer control of the injector set-up. For example, the dual check valve arrangement can be used to allow the syringe to fill automatically from the bottle of contrast or saline, and allows the syringe to push the same to the patient line automatically under the software control of the injector system.

The male luer output ports (12 in FIG. 1a and 11 in FIG. 1b) of the two 3-port tubing sets are connected to the female luer ports (22) of the Y-tubing (20) thus providing a single line contrast or saline output from the two syringes. The female luer ports (22) of the Y-tubing (20) are shown as back check valves with female luer ports, the back check valves functioning to further prevent backflow from the patient line (26).

The patient line (26) has two ends with an input port (28) at one end and an output port (30) at another end. The patient line (26) with a back check valve at each end is connected at one end with input port (28) to the male luer output port (24) of the Y-tubing (20), and connects on the other end through output port (30) to the patient's catheter. The back check valve at each end of the patient line functions to prevent fluid contamination from a patient creeping back into the overall system through the patient line.

For fear of cross-contamination between patients, a prevalent approach in CT scan injections is to do the entire setup for each new patient. This means for each new patient the set-up will require two 3-port tubing sets, one Y-tubing and a patient line. This is an expensive and time consuming setup.

The use of multiple patient dosing or multi-dosing as indicated in the Health Canada study (1996) mentioned above, can reduce the number of times that the entire set-up must be redone. However, even with multi-dosing the set-up is redone on a daily if not hourly basis.

A common problem with current techniques of both single dosing and multi-dosing is the number of connections, the complexity of set-up, the mismatch amongst the components supplied by different vendors and the associated costs involved.

The multiple connections can lead to several problems including, for example, (a) touch contamination, (b) leakage from loose connections or cross-threaded male/female connections, (c) leakage from cracked connections due to too much force being applied when threading male/female connections, and (d) increased inventory costs.

The mismatch in the pressure and flow rating amongst the components supplied by different vendors can lead to blown tubing, blown check valves and leakages. The mismatch in the pressure and flow ratings of the different components can also slow down the speed of contrast delivery and thus effectiveness of the contrast injections. The speed of injection depends on the pressure that the injector applies to push the contrast. This pressure may rise up to 500 psi or higher. If the tubing or the components in its path are not able to tolerate this kind of pressure then parts of it may blow, or come apart or start to leak. The pressure that the tubing can withstand is determined by its hardness (called Durometer) and its input/output diameter (ID/OD). If part of the fluid path can tolerate high pressures while another part can not, then the part which can not tolerate the high pressure will determine the maximum pressure the system can apply. The check valves and the stopcocks are also designed to be high flow or low flow, and can be rated according to tolerated pressure. In an integrated injector components can be selected with compatible pressure and/or flow ratings.

An integrated injector kit is now described that can provide improved connectivity and/or reduced mismatch for both single dosing or multi-dosing contrast injector set-ups. Furthermore, the integrated injector kit can have reduced tubing lengths thereby minimizing circulation volume and wastage of contrast media through tubing lengths.

The integrated injector kit may be particularly advantageous for multi-dosing set-ups as the consequences of connectivity or mismatch problems such as leakages through loose connections or blown check valves may result in cross-contamination of patient fluids. Thus, the integrated injector kit can allow use of the same contrast media container for multiple patients/injections, while maintaining sterility of the contrast agent and preventing patient cross-contamination.

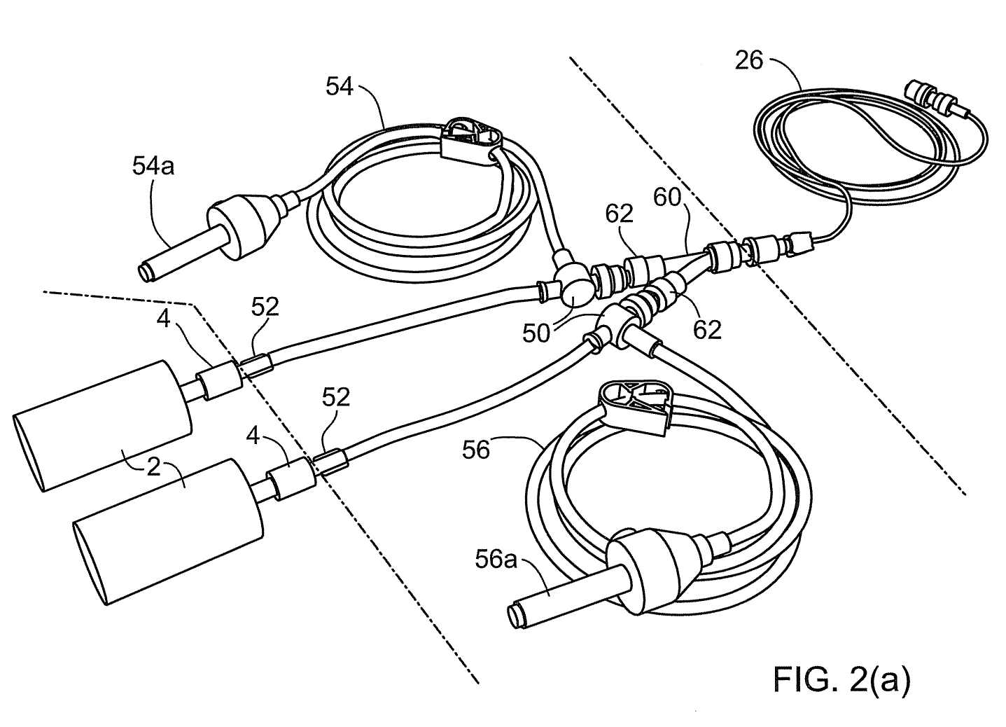

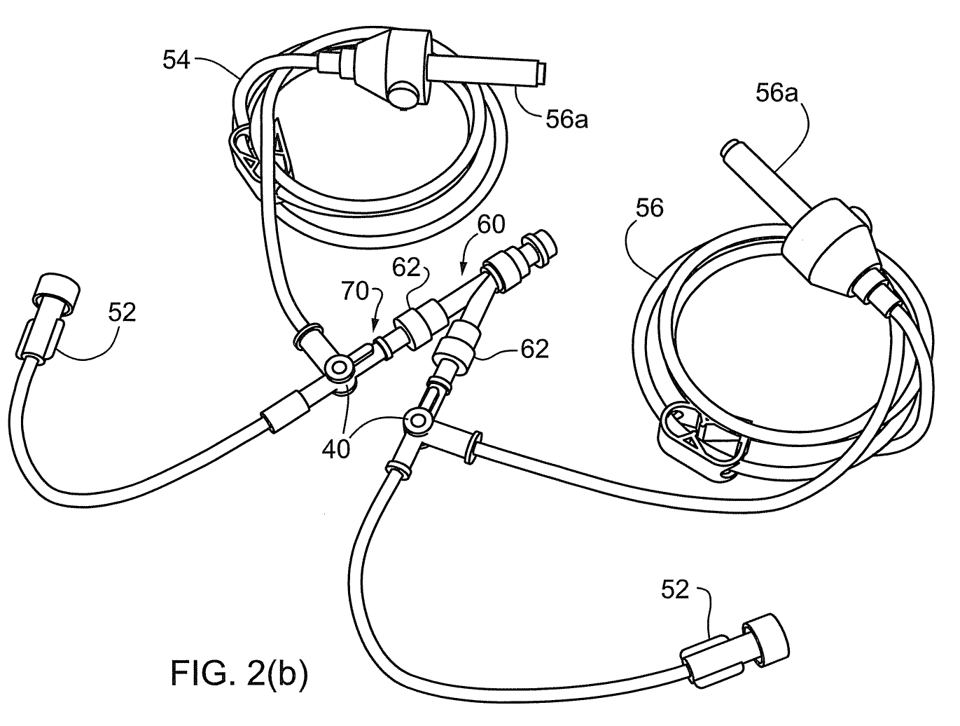

FIGS. 2a and 2b show an integrated injector kit, in which the two syringe 3-port tubing sets and the Y-tubing are pre-assembled into one complete unit and provided to the end user as a kit. The only connections required are: (a) the two connections to the two syringes (2) between the male luer ports (4) of the syringes and the female luer syringe ports (52) of the integrated injector kit; (b) the two connections to the contrast and the saline bags or bottles through the vented spikes (54a, 56a) of the integrated injector kit; and (c) the connection to the patient line (26). In certain examples, the integrated injector kit can further comprise one or both syringes within the pre-assembled complete unit.

Components of the integrated injector kit can be selected to reduce mismatch problems and the pre-assembled complete unit can be tested for loose connectivity or leakages. For example, an assembled injector kit can be tested with air pressure to detect any defects including, without limitation, cracks, loose connections, improper bonding or leakages.

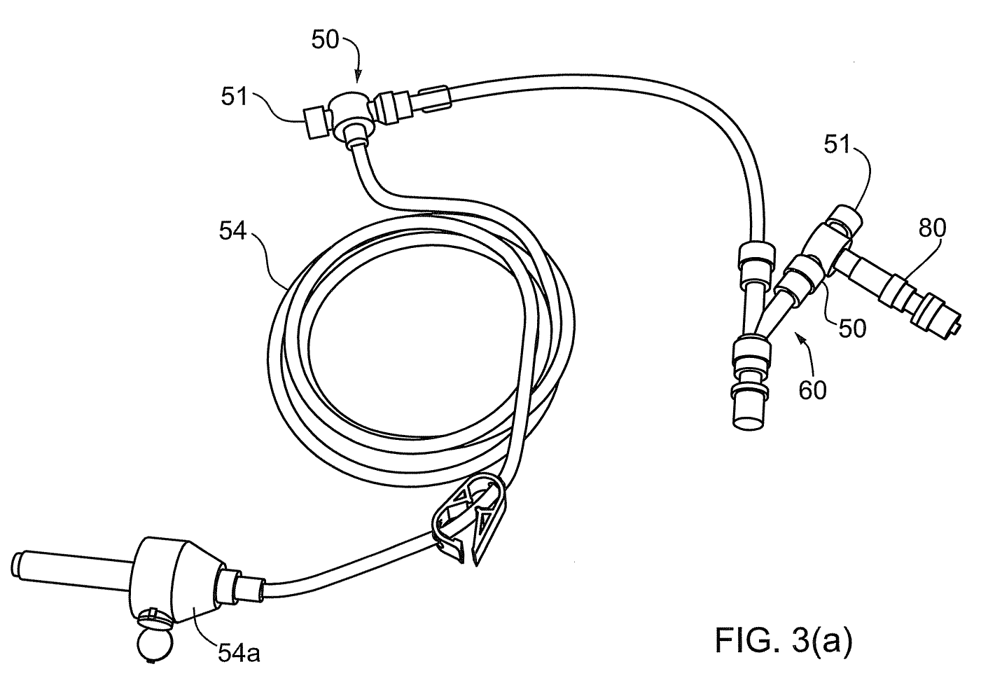

In the integrated injector kit a Y-tubing (60) with reduced tubing length is used. The Y-tubing (60) is an integral part of the kit and therefore no component rating mismatch problems occur. Furthermore, the Y-tubing shown in FIGS. 2a, 2b and 3 has reduced overall tubing lengths thus minimizing the recirculation volume of the contrast and wastage of contrast through the tubing lengths.

FIGS. 2a and 2b show two basic versions of an integrated injector kit that may be used for CT contrast injections. However, other versions are possible, for example, designed with a different type of fluid control valve as described for example in U.S. Pat. Nos. 5,104,387 or 6,238,372.

FIG. 2a shows an integrated injector kit where a 3-port fluid control valve is an automatic dual check valve (50), while in FIG. 2b, the 3-port fluid control valve is a stopcock (40). Both operate in essentially the same manner except that in the stopcock version the operator has to manually turn the stopcock handle to select whether the syringe is filling from the contrast or the contrast is being delivered to the patient. Use of the automatic dual check valve allows for computer control of the integrated injector kit.

In FIG. 2a solid white lines are used to designate the integrated injector kit, with patient line (26) and syringes (2) designated as being outside the integral unit. The integrated injector kit shown in FIG. 2a comprises Y-tubing (60) having an output port that is for releasable connection with patient line (26) and two input ports that each independently and integrally connect with an automatic dual check valve (50). The input ports of the Y-tubing each comprise a back check valve (62) that functions to safeguard against contamination of the media lines. The output port of the Y-tubing can optionally comprise a check valve. Each dual check valve (50) is a 3-port fluid control valve comprising: (a) male luer output port that is integrally connected to back check valve (62); (b) an inlet port for receiving contrast or saline by integral connection with tubing (54, 56) that ends in a vented spike (54a, 56a) that is for releasable connection with a contrast or saline container; and (c) a syringe port integrally connected with tubing that ends in a female luer port (52) that is for releasable connection with the male luer output port (4) of the syringe (2).

In FIG. 2b all of the components shown are integrally connected. The integrated injector kit shown in FIG. 2b comprises Y-tubing (60) having an output port that is for releasable connection with patient line (not shown) and two input ports that are each independently and integrally connected with a stopcock (40). The input ports of the Y-tubing each comprise a back check valve (62) to safeguard against contamination of the media lines. The output port of the Y-tubing can optionally comprise a check valve. Each stopcock (40) is a 3-port manual fluid control valve comprising: (a) male luer output port that is integrally connected to back check valve (62); (b) an inlet port for receiving contrast or saline by integral connection with tubing (54, 56) that ends in a vented spike (54a, 56a) that is for releasable connection with a contrast or saline container (not shown); and (c) a syringe port integrally connected with tubing that ends in a female luer port (52) that is for releasable connection with a male luer output port of a syringe (not shown).

FIG. 3 shows an integrated injector kit that can be used for contrast injection for MRI. In FIG. 3 all of the components shown are integrally connected. The integrated injector kit shown in FIG. 3 comprises Y-tubing (60) having an output port that is for releasable connection with patient line (not shown) and two input ports that are each independently and integrally connected with an automatic dual check valve (50). The Y-tubing does not comprise a back check valve; back check valves may optionally be included in one or more of the two input ports and the output port. Each dual check valve (50) is a 3-port fluid control valve comprising: (a) male luer output port that is integrally connected to the input port of the Y-tubing; (b) an inlet port for receiving contrast or saline by either integral connection with tubing (54) that ends in a vented spike (54a) that is for releasable connection with a saline container (not shown) or integral connection with a male luer adaptor/fitting (80) that is for releasable connection to a contrast agent vial (not shown); and (c) a female luer syringe port (51) that is for releasable connection with a male luer output port of a syringe (not shown).

Optional components of the integrated injector kit will be readily recognized by the skilled person. For example, the tubing extending from the syringe inlet port of the dual check valve (50) or stopcock (40) and ending in female luer port (52) as shown in FIGS. 2a and 2b can be reduced or can be entirely replaced with a connection of the male luer port (4) of syringe (2) directly to the syringe inlet port. As another example, the syringe can optionally be integrally connected with the syringe port of the fluid control valve or with tubing that is extending from the syringe port. As still another example, the tubing (54, 56) shown in FIGS. 2a and 2b to be extending from the fluid control valve (50, 40) to the contrast or saline containers may be reduced or replaced in it entirety. FIG. 3 shows an example where there is no tubing connecting the contrast vial to the fluid control valve. Instead an MRI contrast vial is connected to a male luer adaptor/fitting which is in turn integrally connected with the inlet port of the fluid control valve. As yet another example, a check valve may be integrally connected to the output port of the Y-tubing. As a further example, the fluid path defined by each pair of fluid control valve and input arm of the Y-tubing comprises one back check valve, with further back check valves being optional; FIG. 2a shows back check valves in both the fluid control valves and the input arms of the Y-tubing, FIG. 2b shows a back check valve in the input arms only, and FIG. 3 shows a back check valve in the fluid control valves only. As an even further example, filters for trapping bacteria may be placed in the Y-tubing.

As yet a further example of optional components, While joining the 3 way stop cocks to the back check valves in FIG. 2(b), a male to male luer connector (70) may be added in one of the lines in cases where connection of the female luer port of the back check valve to the male luer port of the stop cock for both input arms of the Y-tubing results in both the saline and the contrast administration lines exiting on the same side which may be inconvenient for an operator. Use of a male/male connector (70) allows the stopcocks to be positioned such that the saline and the contrast administration lines exit on opposite ends as shown in FIG. 2(b). Still other optional modifications will be recognized by the skilled person.

The skilled person will also recognize different methods of achieving a secure and durable integral connection. For example, certain components and connections may be combined to be produced as a single piece by injection molding. As another example, certain components and connections may be achieved through bonding using a medical grade solvent, or ultrasonic bonding or some other form of bonding.

As shown in FIG. 2a and 2b, Y-tubing (60) comprises back check valves that are connected to the dual check valves (50) or to the stopcocks (40). The back check valves of the input arms of the Y-tubing in FIG. 2a are optional because the 3 port syringe tubing already contains a back check valve. However, in FIG. 2b the back check valves of the Y-tubing are desired to achieve protection against backflow of fluid because the stopcocks do not have any back check valves and therefore no protection against backflow.

Since most of the commercially available back check valves and stopcocks are made of polycarbonate elastomer, bonding the back check valves (62) to either the automatic dual check valves (50) or to the stopcocks (40) was tested using several medical grade solvents.

The use of the medical grade solvent, cyclohexanone to join these components caused cracks during the assembly process and thus made the final assembled device leak. The solvent started to attack the plastic. In order to achieve a proper connection the amount of cyclohexanone applied had to be carefully controlled.

Therefore, several medical grade solvents were tested with respect to ease of application and bonding result, for example for their bonding strength, for the amount of solvent required, and for the pressure needed to be applied to obtain a secure bond. Methylethylketone (mek), tetrahydrofuran (thf), and cyclopentanone were found to produce a satisfactory result. Satisfactory bonding was eventually achieved using cyclohexanone by carefully controlling the applied amounts, however it was found to have a long curing time, for example greater than 10 minutes.

While bonding the back check valves (62) to the dual check valve (50), solvent was applied to the female luer port of the back check valve and the male luer lock connection of the dual check valve (50) was then threaded into the female luer port of the back check valve. Generally, when connecting a male luer lock to a female luer lock device, the two are threaded in completely to make a secure connection. However, in this case complete threading of the two components resulted in the tubing for the contrast and for the saline exiting on the same side, instead of coming from the opposite sides as shown in FIG. 2(a). Exiting of the tubing from the same side may inconvenience an operator while connecting the contrast and the saline administration lines to the respective bags or containers. The two lines would tend to tangle up. Therefore, the two components were not threaded in completely, and instead were threaded in to have the contrast and the saline lines come out on the opposite sides, and a suitable solvent was used to achieve a secure and durable connection. Obtaining components from different manufacturers may allow for complete threading while having contrast and saline lines exiting on opposing sides of the injector. However, when complete threading does not produce the desired orientation of exiting lines, then incomplete threading can be used, with a suitable solvent that provides proper bonding strength to achieve a secure and durable connection.

Similarly, in joining the back check valves (62) to the stopcocks (40) in FIG. 2b, solvent was applied to the female luer ports of the back check valves, and the male luer lock connections of the stopcocks were incompletely or partially threaded into the female luer lock ports of the back check valve to achieve a secure and durable connection, while also achieving the desired orientation of contrast and saline lines exiting on opposing sides of the injector.

The injector kit can be packaged with instructions for use of the kit to deliver a contrast agent to a patient including, without limitation, connection of the kit to a patient line, connection of the kit to a contrast media agent container, connection of the kit to a saline container, connection of the kit to injector syringes under specified pressure and/or flow ratings, or use of the kit for multi-dosing.

In use the injector kit can provide a performance advantage. The reliability of the setup is improved. The kit due to reduced number of connections and minimum handling is inherently less prone to touch contamination which is one of the highest concerns of the hospital infection control departments all over. Furthermore, the kit due to a properly bonded one piece structure is much more resistant to any leakages or cracks caused by improper or overly tightened connections, which again minimizes the possibility of contaminating the contrast media. Efficiency and effectiveness of the contrast delivery may be improved as well because the pressure and flow ratings of all components in the kit can be matched to specified ratings which is not always possible to guarantee in a setup of screwed-in individual components as there does not exist a harmonized compatibility standard for interfacing them.

The above-described embodiments are intended to be examples and alterations and modifications may be effected thereto, by those of skill in the art, without departing from the scope of the invention which is defined by the claims appended hereto.

What is claimed is:

1. An integrated contrast injector kit comprising:
Y-tubing comprising an output arm that is for releasable connection with a patient line and first and second input arms that each independently and integrally connect with an output port of first and second fluid control valves;
each fluid control valve comprising: (a) the output port that is integrally connected to one of the first and second input arms of the Y-tubing; (b) an inlet port for connection with a container of a contrast agent or saline and for receiving the contrast agent or saline; and (c) a syringe port for connection with a syringe; and
each pairing of fluid control valve and input arm of the Y-tubing defining a fluid path comprising at least one back check valve.

2. The integrated contrast injector kit of claim 1, wherein the first input arm of the Y-tubing comprises a first back check valve and the second input arm of the Y-tubing comprises a second back check valve.

3. The integrated contrast injector kit of claim 2, wherein the first and second back check valves are located at the beginning of the first and second input arms and each is integrally connected with the output port of the first and second fluid control valves.

4. The integrated contrast injector kit of claim 1, wherein the output arm of the Y-tubing comprises a back check valve.

5. The integrated contrast injector kit of claim 4, wherein the back check valve is located at the end of the output arm of the Y-tubing and is for releasable connection with the patient line.

6. The integrated contrast injector kit of claim 1, wherein the inlet port of at least one of the first and second fluid control valves is integrally connected to tubing that ends in a vented spike that is for releasable connection with the contrast agent or saline container.

7. The integrated contrast injector kit of claim 1, wherein the syringe port of at least one of the first and second fluid control valves are integrally connected with tubing that ends in a female luer port that is for connection with the syringe.

8. The integrated contrast injector kit of claim 1, wherein the syringe port or the tubing extending therefrom is for releasable connection with the syringe.

9. The integrated contrast injector kit of claim 1, further comprising the syringe integrally connected to the syringe port, or tubing extending therefrom, of at least one of the first and second fluid control valves.

10. The integrated contrast injector kit of claim 1, wherein the fluid control valve is a 3-port manual or automatic fluid control valve.

11. The integrated contrast injector kit of claim 1, wherein the fluid control valve is an automatic dual check valve.

12. The integrated contrast injector kit of claim 1, wherein the fluid control valve is a stopcock.

13. The integrated contrast injector kit of claim 1, wherein integrally connected components are bonded using a medical grade solvent and/or using a welding technique.

14. The integrated contrast injector kit of claim 13, wherein the welding technique is ultrasonic welding or laser welding.

15. A method for delivering a contrast agent to a subject, comprising employing an integrated contrast injector kit of claim 1 to deliver a contrast agent to a subject.

16. A method for delivering multi-dose contrast agent injections to patients, the method comprising employing an integrated contrast injector kit of claim 1 to deliver multi-dose contrast agent injections to patients.

17. The method of claim 15, wherein the contrast agent is for MRI, CT, or ultrasound imaging.

18. The method of claim 16, wherein the contrast agent is for MRI, CT, or ultrasound imaging.
